(19) United States
(12) Patent Application Publication
(10) Pub. No.: US 2026/0328786 A1
(43) Pub. Date: Oct. 22, 2026

(54) SAMPLE CONTAMINATION DETECTION APPARATUS AND METHODS

(71) Applicant: Siemens Healthcare Diagnostics Inc., Tarrytown, NY (US)

(72) Inventors: Thomas Kampfrath, White Plains, NY (US); Jennifer Snyder, Newark, DE (US)

(73) Assignee: Siemens Healthcare Diagnostics Inc., Tarrytown, NY (US)

(21) Appl. No.: 19/099,618

(22) PCT Filed: Aug. 14, 2023
(86) PCT No.: PCT/US2023/072132

§ 371 Date: Jan. 29, 2025

Related U.S. Application Data
(60) Provisional application No. 63/375,204, filed on Sep. 9, 2022.

(51) Int. Cl. G01N 33/84; G01N 33/66; G01N 33/70

(52) U.S. Cl. CPC G01N 33/84; G01N 33/66; G01N 33/70

(57) ABSTRACT

Disclosed is a blood contamination detection apparatus. The blood contamination detection apparatus comprises a computer-based contamination determining module configured to receive: one or more baseline quantified test results on an uncontaminated blood sample obtained from a patient; one or more quantified test results for one or more analytes of a blood sample or a blood component; and data input on a type of IV fluid infusion that the patient has received that is able to affect the one or more quantified test results. The computer-based contamination determining module comprises a contamination determining routine executable on a computer and configured to compare differences between the one or more baseline quantified test results and the one or more quantified test results against preset threshold difference values and/or directions and provide a contamination output. Sample contamination detection method are provided, as are other aspects.

### CROSS REFERENCE TO RELATED APPLICATIONS/INCORPORATION BY REFERENCE STATEMENT

This application claims benefit under 35 USC § 119(e) of U.S. Provisional Application No. 63/375,204, filed Sep. 9, 2022. The entire contents of the above-referenced patent application are hereby expressly incorporated herein by reference.

### FIELD

The present disclosure relates to apparatus and methods of determining intravenous (IV) fluid contamination of patient samples, such as in drawn whole blood and blood components thereof.

### BACKGROUND

In phlebotomy, a sample of whole blood (“blood sample”) is drawn from a patient. The blood sample is typically drawn in order to run a panel of blood chemistry tests on the blood sample to assist in making disease diagnosis and/or treatment. Such testing may be carried out many times a day or over a series of days as the patient undergoes diagnosis and/or treatment in a healthcare facility.

Many hospitalized patients continuously receive intravenous fluid (IV fluid) infusions of various kinds to treat an underlying medical condition. There is a move underway in the hospital setting to minimize the number of needle sticks that a patient has to endure. Thus, sometimes the blood sample is obtained directly from an IV catheter that has already been inserted into the patient. The patient may be or has been receiving one or more IV fluids through the IV catheter.

Clinical & Laboratory Standards Institute (CLSI) guidelines (e.g., CLSI GP41) provide suggestions for “Collection of Diagnostic Venous Blood Specimens.” The guidelines note that there is risk of obtaining erroneous and misleading results when drawing blood from a patient with an IV catheter. The guidelines recommend using the opposite arm (not the arm with the IV catheter) whenever possible, and when not possible, collecting the specimen in the arm with the IV, but at a location below the IV catheter site. CLSI guidelines further provide recommendations about techniques for drawing blood from below an IV catheter, recommending that the IV be turned off for a period of two minutes or longer and that a tourniquet be placed between the IV site and the blood draw site. Finally, the guidelines mention collection above the IV catheter is not recommended and should be done only when all other options for blood collection have been exhausted.

### SUMMARY

Some embodiments of the present disclosure provide a blood contamination detection method. In particular, the method comprises obtaining a blood sample from a location proximate to an IV catheter as a patient is undergoing or has undergone an IV fluid infusion; analyzing the blood sample or a blood component thereof to provide one or more quantified test results for one or more analytes of the blood sample; obtaining an uncontaminated blood sample from the patient; analyzing the uncontaminated blood sample or an uncontaminated blood component thereof to provide one or more baseline quantified test results for the one or more analytes in the uncontaminated blood sample or the uncontaminated blood component; obtaining data input on a type of infusion that the patient has received that is able to affect the one or more quantified test results; analyzing, with a computer-based contamination determining module containing a contamination determining routine executable on a computer, differences between the one or more quantified test results and the one or more baseline quantified test results; determining contamination of the blood sample with the contamination determining routine based upon the differences as compared to preset threshold values; and providing a contamination output.

Some embodiments of the present disclosure provide an apparatus configured to analyze a blood specimen or a component thereof obtained from a patient. The blood contamination detection apparatus comprises a computer-based contamination determining module configured to receive: one or more baseline quantified test results on an uncontaminated blood sample obtained from a patient; one or more quantified test results for one or more analytes of a blood sample or a blood component; and data input on a type of IV fluid infusion that the patient has received that is able to affect the one or more quantified test results, and wherein the computer-based contamination determining module comprises a contamination determining routine executable on a computer and configured to compare differences between the one or more baseline quantified test results and the one or more quantified test results against preset threshold difference values and provide a contamination output.

Still other aspects, features, and advantages of the present disclosure may be readily apparent from the following detailed description by illustrating a number of example embodiments and implementations. The present disclosure may also be capable of other and different embodiments, and its several details may be modified in various respects, all without departing from the scope of the present invention. Further features and aspects of embodiments will become more fully apparent from the following detailed description, the claims, and the accompanying drawings. Accordingly, the drawings and description are to be regarded as illustrative in nature, and not as restrictive. Any feature of the various embodiments described herein can be used in any of the other embodiments described herein, as applicable. The disclosure is to cover all modifications, equivalents, and alternatives falling within the scope of the claims and their equivalents.

### BRIEF DESCRIPTION OF THE DRAWINGS

The accompanying drawings, described below, are for illustrative purposes and are not necessarily drawn to scale. The drawings are not intended to limit the scope of the disclosure in any way. Like numerals are used throughout the specification and drawings to denote like elements.

FIG. 1 illustrates a partial cross-sectioned view of an IV catheter (cannula) inserted in an patient's extremity and used to obtain a blood sample as the patient is undergoing or has recently undergone an IV infusion of a IV fluid.

FIG. 2 illustrates a schematic view of a blood contamination detection apparatus containing a contamination determining module according to one or more embodiments of the disclosure.

FIG. 3 illustrates a flowchart of a method of determining contamination of a blood sample according to embodiments of the disclosure.

### DETAILED DESCRIPTION

In some blood analysis procedures, such as when conducting tests on drawn whole blood or a blood component processed therefrom (e.g., serum or plasma) as described above, it may be desirable to understand, when reviewing the results of the particular test, whether the drawn blood specimen may have been contaminated in some way by an IV fluid infusion. For example, common types of intravenous (IV) fluids that can be administered via an IV catheter (via a cannula) include isotonic, hypotonic, and hypertonic IV fluids. Such infusions can include various crystalloid solutions containing small dissolved solids molecules that may pass easily into the bloodstream, as well as colloid solutions. Examples of common crystalloid solutions include: 1) normal saline, which is 0.9% salt (NaCl) in water, 2) half saline, which is 0.45% salt (NaCl) in water, 3) D 5W which is 5% dextrose in water, and 4) lactated ringer's solution (LR). Other types of IV fluids may also be administered by an IV catheter. Blood collections on patients undergoing IV fluid infusions may be performed downstream of the IV port or directly from the line, which, to various degrees, can lead to IV fluid contamination of the collected blood. One study has shown this issue along with other ordering and sample collection errors can contributes to approximately 46.0%-68.2% of errors in the total laboratory testing process (See Plebani, et al. 2010). Such IV fluid contamination can alter patient results with potential for mistreatment or requests for sample recollection associated with potential for delays and increased costs.

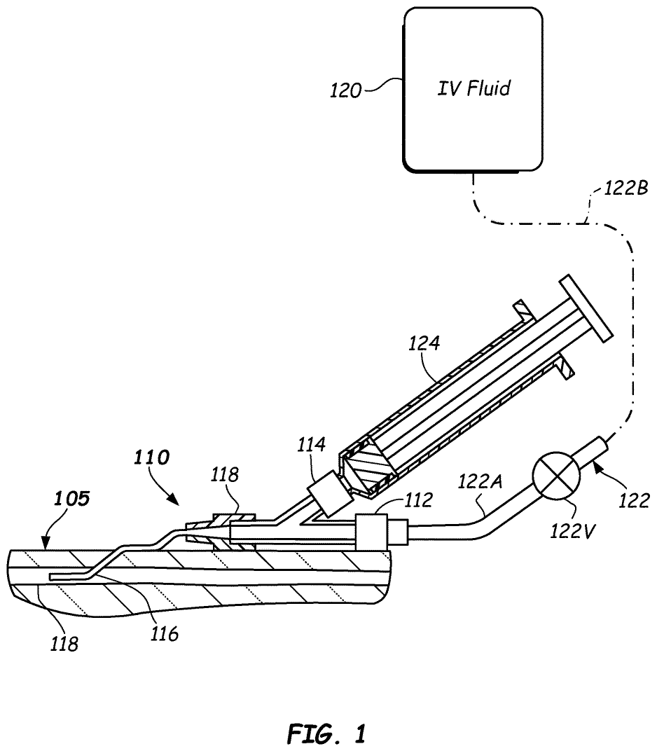

In FIG. 1, a portion of patient's extremity 105 such as a hand, arm, neck, or the like, is shown. An IV catheter 110 has been installed, otherwise known as a peripheral intravenous catheter (PIVC) or IV port. The IV catheter 110 is a dual type having a first connection 112 for attachment to an intravenous infusion source (IV fluid 120), and a second connection 114 that may be used to draw a blood sample (e.g., with a suitable syringe or other suitable collection vessel) from the patient without having to undo the first connection 112. Although, an IV catheter 110 is shown, the present disclosure is equally applicable for use with peripherally inserted central catheters (PICC lines), where the line can be located somewhere above the elbow in either the cephalic vein, basilic vein, or brachial vein. Furthermore, the present disclosure is equally applicable to the use of IV catheters having more than two connections.

In more detail, the IV catheter 110 can include a cannula 116 that is inserted in a suitable blood vessel 118, such as the cephalic vein, basilic vein, or the median cubital vein. In some cases a needle may be used in place of a cannula 116. Other veins may be used as well. When drawing a blood sample from the second connection 114 of the IV catheter 110 certain procedures are supposed to be followed by the medical professional (e.g., phlebotomist or nurse).

In particular when a blood sample is drawn, via a blood collection vessel 124 (e.g., syringe or other blood collection device or tube), while a volume of the IV fluid 120 is attached to the first connection 112 via a suitable conduit 122, the flow of the IV fluid 120 is supposed to be shut off for a defined period of time. Further, a volume of waste fluid should be drawn off and removed before obtaining the blood sample from the IV catheter 110. However, such procedures may not be done or may only be partially done (incompletely followed) either though incomplete training, attempting to reduce wastage of blood with the goal to prevent hospital acquired anemia, or simply forgetting procedures.

For example, the medical professional may not wait long enough after turning off the flow of IV fluid 120 to the IV catheter 110, the waste fluid may not be drawn off or it may be insufficiently drawn off, or the valve 122V between the IV fluid 120 and the first connection 112 may be only partially closed so that some IV fluid 120 may still be flowing as the blood sample is obtained. As such, the blood sample obtained may possibly be contaminated to some extent with IV fluid 120 that is or has been recently infused.

Accordingly, there is a need to coordinate the process of drawing the blood sample with a contamination diagnostic method that will allow the professional reading the various quantified test results from a laboratory information system (LIS) or hospital information system (HIS) to better understand whether then test sample was possibly contaminated. The output from the IV contamination determining method and IV contamination determining apparatus may be provided as an output of probability of contamination of the blood sample, for example. The methods and apparatus can automatically detect IV fluid contamination in a patient blood sample based on direction and/or magnitude of result changes for many commonly used IV fluid types. Thus, the medical professional can make an informed judgement as to the correctness and believability of the one or more quantified test results, given that it was taken in the proximity of an IV catheter 110 that had been or is undergoing an infusion of an IV fluid 120.

These and other aspects and features of the present disclosure will be described with reference to FIGS. 1-3 herein.

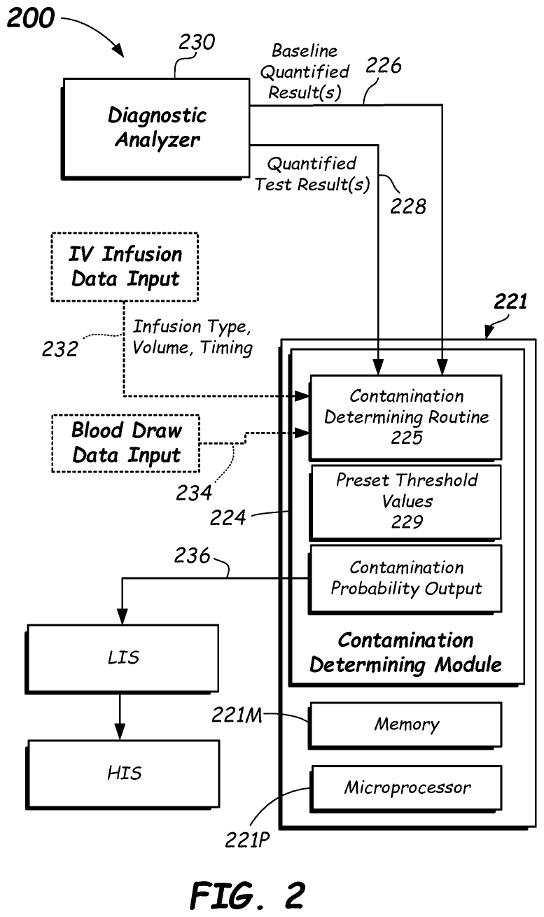

In accordance with a first embodiment of the disclosure, as best shown in FIG. 2, a blood contamination detection apparatus 200 is shown. The blood contamination detection apparatus 200 comprises a contamination determining module 224 that is computer based and can be stored in a computer memory 221M and is executable on a microprocessor 221P of a computer 221 or other processing device. The contamination determining module 224 may be a software program configured to carry out the contamination determination based upon various inputs to the module 224. In particular, the contamination determining module 224 contains a contamination determining routine 225 that is configured to receive data inputs of one or more baseline quantified test results 226 for one or more analytes contained in an uncontaminated blood sample obtained from a patient, and one or more quantified test results 228 for one or more analytes of a blood sample or a blood component thereof. The blood sample may be whole blood. A blood component thereof can be serum or plasma, for example.

The uncontaminated blood sample obtained from the patient to be used for obtaining the one or more baseline quantified results 226 can be obtained preferably before the blood sample from which the one or more quantified test results 228 are determined and for which contamination is to be determined. The uncontaminated blood sample can be taken from an extremity, e.g., an arm that is not undergoing an IV infusion, before any IV infusion, or at a time before or after the IV infusion has been completed, or by other means where it is clear that the blood sample will be uncontaminated with IV fluid, such as upon initial admission to the medical facility and/or before any IV infusion has taken place.

The one or more quantified test results 228 for one or more analytes of a blood sample or a blood component are obtained by testing, via the diagnostic analyzer 230, one or more blood samples or blood components thereof, wherein the blood sample was obtained from proximate to the IV catheter 110, such as directly from the second connection 114 (FIG. 1). These one or more quantified test results 228 are at risk of contamination for the various reasons described herein, such as taking the blood sample too soon in time after the IV infusion (so that the veins in the proximity contain IV solution, by not drawing off sufficient waste fluid from the IV catheter, and/or by not fully shutting off the flow of the IV fluid infusion.

The various inputs to the contamination determining routine 225 of the contamination determining module 224 can be from any suitable diagnostic analyzer 230, such as a clinical chemistry analyzer or other suitable assay instrument that is operable to quantify one or more analytes of interest contained in the blood sample or the blood component thereof. The one or more analytes tested for can comprise glucose, sodium (Na+), chloride (Cl—), potassium (K+), calcium (Ca2+), lactate creatinine, and BUN, for example. Other analytes of a basic or comprehensive metabolic panel may be tested as well.

The various inputs to the contamination determining routine 225 of the module 224 can further optionally include IV infusion data input 232. The IV infusion data input 232 can comprise a type of IV fluid infusion that the patient has received. In some instances, the type of IV fluid infusion is of a chemical composition that can be able to affect the one or more quantified test results 228. Thus, knowing the type of IV fluid infusion can help assist in making a contamination determination. For example, the IV infusion data input 232 on infusion type can be selected from a drop down menu and/or otherwise input by the medical professional or automatically, such as by a barcode entry from a barcode associated with the IV Fluid 210.

The IV infusion types may be selected from the group of saline-containing, dextrose-containing, and lactate-containing IV fluids. Saline-containing IV infusions can include normal saline, which is an isotonic solution that contains 0.9% NaCl, half normal saline, which is a hypotonic IV solution and contains 0.45% NaCl, and 3% saline, which is a hypertonic solution and contains 3% NaCl. Dextrose-containing IV infusions can include D 5W, which is 5% dextrose in water. Lactate-containing IV fluids such as lactated ringers infusions contain sodium, chloride, potassium, calcium, and lactate. Standard ringers lactate contains 130 mmol/L Na, 109 mmol/L Cl, 4 mmol/L K3 mmol/L Ca and 28 mmol/L lactate.

Some IV fluid infusions can include combinations of saline and dextrose, such as 5% dextrose and 0.45% sodium chloride. Other IV fluid infusions can include combinations of dextrose and lactated ringers, such as 5% dextrose and lactated ringers. Other IV Fluid makeups could be provided as IV data input 232.

The IV data input 232 can, in some embodiments, also comprise the time of starting the IV infusion as well as the volume of the IV fluid infused into the patient. The IV data input 232 can also comprise a rate of intake (flow rate) of the IV fluid infusion and/or the time of stopping the IV infusion. These data inputs can be entered manually as IV infusion data input 232 by the medical professional or may be provided as a data input directly from an electronic infusion device (EID).

In some embodiments, the IV infusion data input 232 can include one or more of the time of starting the IV fluid, the volume of IV fluid infused, the rate of intake (flow rate) of the IV fluid by the patient, and the time of stopping the IV fluid infusion.

The input to the contamination determining routine 225 can optionally further include blood draw data input 234. These data can include the time of draw of the blood sample, and even the volume of blood drawn. The identity of the medical professional obtaining the blood sample from the IV catheter 110 can also be a blood draw data input 234. Should a high value of contamination probability output 236 from the contamination determining routine 225 be output routinely for a particular medical professional, and then some sort of notification may be output and thus further training may be undertaken, or other measure taken to rectify this.

The computer-based, contamination determining module 224 comprises a contamination determining routine 225 that is stored in a suitable memory 221M is executable on a microprocessor 221P of a computer 221. For example, the computer 221 may be a middleware computer that is configured to perform other functions, such as the operation of a laboratory carrying out the analyte testing, routing of blood samples therein, and routing of results to the laboratory information system (LIS) and hospital information system (HIS). The contamination determining routine 225 is configured to compare differences between the one or more baseline quantified test results 226 and the one or more quantified test results 228. In particular, based on these differences and or directions, a contamination output may be generated, such as a probability of contamination.

For example, the differences between the one or more baseline quantified test results 226 and the one or more quantified test results 228 can be compared against preset threshold difference values and/or directions and provide a contamination output, such as a contamination probability output 236. The contamination probability output 236 can be expressed as a numerical output (such as a percentage such as ranging from 0% to 100%) or simply an output of a HIGH, MODERATE, LOW probability. Thus, the output can alert the medical professional reviewing the one or more quantified test results 228 what credence to place on them based on the probability they have been contaminated by an IV fluid infusion. In cases where input of the type of IV fluid is not provided, the output may predict the type of IV fluid infusion that took place. This, in turn, could then be verified by the medical professional performing a selected patient chart review for the patient and comparison to the IV fluid as documented in the patient's chart.

In some embodiments, the one or more quantified test results may be flagged in the laboratory information system (LIS) and/or the hospital information system (HIS) with a warning that the one or more quantified test results 228 have a defined probability of IV fluid contamination.

The one or more baseline quantified test results 226 may be for tests quantifying analytes such as glucose, sodium, chlorine, potassium, calcium, lactate, creatinine, and/or BUN. Other data input for the one or more baseline quantified test results 226 can include fasting condition, i.e., number of hours after eating or last glucose infusion.

Likewise, the one or more quantified test results 226 for which contamination is to be determined may be for tests quantifying the analytes glucose, sodium, chlorine, potassium, calcium, lactate, creatinine, and. The quantified test results 226 can be for the same analytes tested for the one or more baseline quantified test results 226 or a subset thereof.

The differences between the one or more baseline quantified test results 226 and the one or more quantified test results 228 can be compared against preset threshold difference values to determine the contamination probability. These preset threshold values 229 can be difference values for each of the respective analytes and can be established experimentally. For example, certain preset threshold differences in mmol/L can be associated with different contamination probability percentages. For example, 0%-20% can be associated with a first difference range, 21%-40% probability can be associated with a second difference range, 41%-60% probability can be associated with a third difference range, 61%-80% probability can be associated with a fourth difference range, and 81%-100% probability can be associated with a fifth difference range. Look-up tables for the various analytes can be prepared based on experimental result testing. The preset threshold difference values associated with the various % probabilities of contamination can be preset individually for each of the one or more analytes, or aggregated for the entire sample. 100% is associated with the maximum attainable contamination probability, whereas 0% can be associated with no contamination. For example, each of the possible contamination contributors (taking sample too soon after shutting off IV fluid flow, improper flush, improper flow shutoff at valve) can be individually tested and a contamination probability values determined from the results.

One experiment may involve varying the time of obtaining the blood sample in increments from immediately after turning off the IV infusion an up to about four minutes after. Another experiment may involve varying the amount of waste fluid drawn off in increments before obtaining the sample. Another may involve improper shutting off of the infusion flow. Combinations of the aforementioned may also be tested.

Given that analyte levels can change over time due to intake of fluid, food, medicines by the patient, it is desired that the baseline test sample be relatively fresh, i.e., obtained not too far before the threshold blood sample to be quantified for contamination by IV fluid infusion is obtained. One preferred method of obtaining the baseline sample is by obtaining the blood sample from the IV catheter 110 before administering the IV infusion.

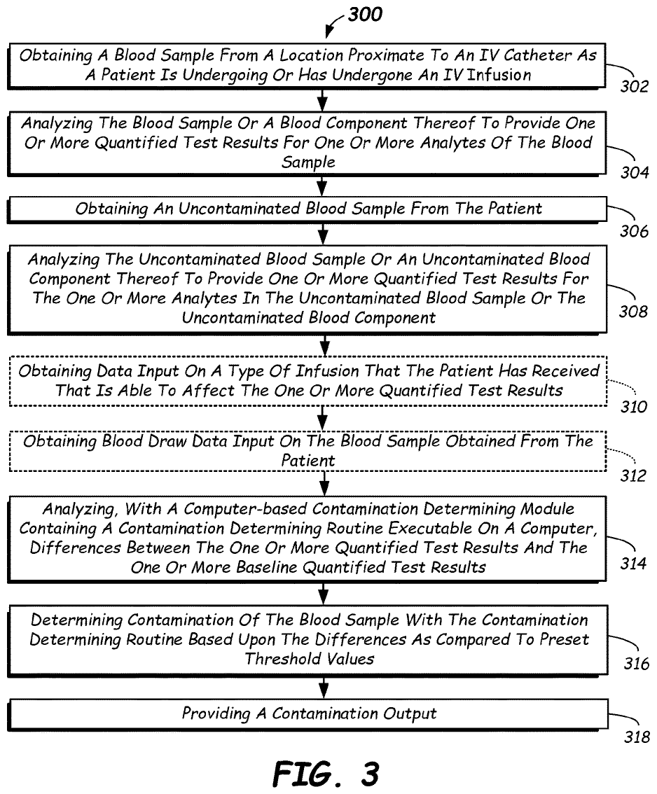

According to another aspect of the present disclosure, a method of determining contamination of a blood sample is provided. The sample contamination detection method 300 is best shown in the flowchart of FIG. 3. The sample contamination detection method 300 comprises, in block 302, obtaining a blood sample from a location proximate to an IV catheter as a patient is undergoing or has undergone an IV fluid infusion. For example, the IV fluid infusion of IV fluid 120 may be shut off via the valve 122V located between conduit sections 122A and 122B (FIG. 1) prior to obtaining the blood sample. The blood sample may be taken from the second connection 114 by a blood collection vessel 124 via routine methods.

The sample contamination detection method 300 further comprises, in block 304, analyzing the blood sample or a blood component thereof (e.g., serum or plasma obtained via centrifugation of the blood sample) to provide one or more quantified test results for one or more analytes of the blood sample or the blood component. The quantified test results 226 can be measurements of amounts of one or more analytes in the blood sample or a blood component (e.g., serum or plasma) thereof, such as a glucose level, a sodium level, a chloride level, a potassium level, a calcium level, a lactate level, or the like. Analysis can be by any suitable diagnostic analyzer 230.

Sample contamination detection method 300 further comprises, in block 306, obtaining an uncontaminated blood sample from the patient, and, in block 308, analyzing the uncontaminated blood sample or an uncontaminated blood component thereof (e.g., serum or plasma) to provide one or more quantified test results for the one or more analytes in the uncontaminated blood sample or the uncontaminated blood component. The uncontaminated blood sample is obtained in a manner where it is unaffected by any IV fluid infusion. The uncontaminated blood sample can be obtained before the IV infusion starts and can be obtained in close proximity in time prior to obtaining the blood sample to be tested for IV fluid contamination.

Sample contamination detection method 300 can further optionally comprise, in block 310, obtaining data input 232 on a type of IV fluid 120 infusion that the patient has received that is able to affect the one or more quantified test results 228. For example, if the analyte of interest is glucose, the type and amount of IV fluid 120 containing dextrose may be a relevant input. If the analyte of interest is sodium (Na+) then whether the patient has received a saline-containing IV fluid infusion may be relevant. Likewise, if the analyte of interest is chlorine (Cl—) then whether the patient has received a saline-containing IV fluid infusion may also be relevant. Similarly, if the analyte of interest is lactate, potassium (K+), or calcium (Ca2+), then whether the patient has received a ringers lactate IV infusion may be relevant.

Thus, it should be recognized that, if the blood draw is improperly done, saline-containing IV fluid infusions can affect Na+ and Cl+ concentrations, and dextrose-containing IV fluid infusions can affect glucose concentrations, for example.

Sample contamination detection method 300 can further optionally comprise, in block 312, obtaining blood draw data input 234 on the blood sample obtained from the patient. This data 234 may include the time of obtaining the blood sample from the patient, the number and type of blood collection vials, drawn blood volume information, and/or details regarding fasting or last food input.

In block 314, the sample contamination detection method 300 further comprises analyzing, with a computer-based contamination determining module (e.g., contamination determining module 224) containing a contamination determining routine (e.g., contamination determining routine 225) executable on a computer (e.g., computer 221), differences between the one or more quantified test results 228 and the one or more baseline quantified test results 226. The analyte specific direction of the differences can also be assessed for IV fluid contamination detection.

In block 316, the method 300 comprises determining contamination of the blood sample with the contamination determining routine based upon the differences as compared to preset threshold values. In other words, the differences between the quantified test results 228 as compared to the baseline test results 226 for each of the corresponding analytes tested can be quantified and then compared to preset threshold values for each analyte of interest. Moreover, a direction of the change can also be determined.

In block 318, the method 300 comprises providing a contamination output. The contamination output 236 can be a contamination probability. For example, the contamination probability can comprise a HIGH or LOW probability or even a MEDIUM probability of contamination or a percentage probability such as 0% to 20%, 21% to 40%, 41% to 60%, 61% to 80%, or 81% to 100%. The probabilities can be determined via reference to a lookup table stored in memory 221M that is established experimentally and contains the threshold differences corresponding to each probability for each analyte.

### NON-LIMITING ILLUSTRATIVE EMBODIMENTS

The following is a list of non-limiting illustrative embodiments of the inventive concept disclosed herein:

Illustrative embodiment 1. A sample contamination detection method, comprising obtaining a blood sample from a location proximate to an IV catheter as a patient is undergoing or has undergone an IV fluid infusion, analyzing the blood sample or a blood component thereof to provide one or more quantified test results for one or more analytes of the blood sample, obtaining an uncontaminated blood sample from the patient, analyzing the uncontaminated blood sample or an uncontaminated blood component thereof to provide one or more baseline quantified test results for the one or more analytes in the uncontaminated blood sample or the uncontaminated blood component, obtaining data input on a type of infusion that the patient has received that is able to affect the one or more quantified test results, analyzing, with a computer-based contamination determining module containing a contamination determining routine executable on a computer, differences between the one or more quantified test results and the one or more baseline quantified test results, and determining contamination of the blood sample with the contamination determining routine based upon the differences as compared to preset threshold values, and providing a contamination output.

Illustrative embodiment 2. The method of illustrative embodiment 1, wherein the contamination output is a contamination probability.

Illustrative embodiment 3. The method according to one of the preceding embodiments, wherein the contamination probability comprises HIGH or LOW or a percentage.

Illustrative embodiment 4. The method according to one of the preceding embodiments, comprising flagging the one or more quantified test results with a warning that the one or more quantified test results have a defined probability of IV fluid contamination.

Illustrative embodiment 5. The method according to one of the preceding embodiments, wherein the one or more quantified test results comprise a blood glucose level.

Illustrative embodiment 6. The method according to one of the preceding embodiments, wherein the analyzing is of the blood component, and the blood component comprises serum or plasma.

Illustrative embodiment 7. The method according to one of the preceding embodiments, wherein the one or more quantified test results comprise a sodium level.

Illustrative embodiment 8. The method according to one of the preceding embodiments, wherein the one or more quantified test results comprise a chloride level.

Illustrative embodiment 9. The method according to one of the preceding embodiments, wherein the obtaining of the blood sample is directly from the IV catheter.

Illustrative embodiment 10. The method according to one of the preceding embodiments, wherein the uncontaminated blood sample obtained from the patient is obtained from a venous puncture unassociated with the IV fluid infusion or prior to a start of the IV fluid infusion.

Illustrative embodiment 11. The method according to one of the preceding embodiments, wherein a type of the IV fluid infusion is selected from a group of: saline-containing solutions, dextrose-containing solutions, and lactate-containing solutions.

Illustrative embodiment 12. The method according to one of the preceding embodiments, wherein a type of infusion is selected from a group of: normal saline, which is 0.9% NaCl in water, half saline, which is 0.45% NaCl in water, D 5W which is 5% dextrose in water, and lactated ringer's solution (LR).

Illustrative embodiment 13. The method according to one of the preceding embodiments, wherein the data input on the IV fluid infusion is a volume of the IV fluid infusion.

Illustrative embodiment 14. The method according to one of the preceding embodiments, wherein the data input on the IV fluid infusion is a start time of the IV fluid infusion.

Illustrative embodiment 15. A blood contamination detection apparatus, comprising a computer-based contamination determining module configured to receive: one or more baseline quantified test results on an uncontaminated blood sample obtained from a patient, and one or more quantified test results for one or more analytes of a blood sample or a blood component, and data input on a type of IV fluid infusion that the patient has received that is able to affect the one or more quantified test results, and wherein the computer-based contamination determining module comprises a contamination determining routine executable on a computer and configured to compare differences between the one or more baseline quantified test results and the one or more quantified test results against preset threshold difference values and provide a contamination output.

Illustrative embodiment 16. The blood contamination detection apparatus according to one of the preceding embodiments, wherein the preset threshold difference values are preset individually for each of the one or more analytes.

Illustrative embodiment 17. The blood contamination detection apparatus according to one of the preceding embodiments, wherein the one or more analytes comprises glucose, sodium, chlorine, potassium, calcium, lactate, creatinine, and/or BUN.

Illustrative embodiment 18. The blood contamination detection apparatus according to one of the preceding embodiments, wherein the contamination output provides a contamination probability.

While some embodiments of this disclosure have been disclosed in example forms, many modifications, additions, and deletions can be made therein without departing from the scope of this disclosure, as set forth in the claims and their equivalents.

What is claimed is:

1. A sample contamination detection method, comprising:
obtaining a blood sample from a location proximate to an IV catheter as a patient is undergoing or has undergone an IV fluid infusion;
analyzing the blood sample or a blood component thereof to provide one or more quantified test results for one or more analytes of the blood sample;
obtaining an uncontaminated blood sample from the patient;
analyzing the uncontaminated blood sample or an uncontaminated blood component thereof to provide one or more baseline quantified test results for the one or more analytes in the uncontaminated blood sample or the uncontaminated blood component;
obtaining data input on a type of infusion that the patient has received that is able to affect the one or more quantified test results;
analyzing, with a computer-based contamination determining module containing a contamination determining routine executable on a computer, differences between the one or more quantified test results and the one or more baseline quantified test results; and
determining contamination of the blood sample with the contamination determining routine based upon the differences as compared to preset threshold values; and
providing a contamination output.

2. The method of claim 1, wherein the contamination output is a contamination probability.

3. The method of claim 2, wherein the contamination probability comprises HIGH or LOW or a percentage.

4. The method of claim 1, comprising flagging the one or more quantified test results with a warning that the one or more quantified test results have a defined probability of IV fluid contamination.

5. The method of claim 1, wherein the one or more quantified test results comprise a blood glucose level.

6. The method of claim 1, wherein the analyzing is of the blood component, and the blood component comprises serum or plasma.

7. The method of claim 1, wherein the one or more quantified test results comprise a sodium level.

8. The method of claim 1, wherein the one or more quantified test results comprise a chloride level.

9. The method of claim 1, wherein the obtaining of the blood sample is directly from the IV catheter.

10. The method of claim 1, wherein the uncontaminated blood sample obtained from the patient is obtained from a venous puncture unassociated with the IV fluid infusion or prior to a start of the IV fluid infusion.

11. The method of claim 1, wherein a type of the IV fluid infusion is selected from a group of:
saline-containing solutions,
dextrose-containing solutions, and
lactate-containing solutions.

12. The method of claim 1, wherein a type of infusion is selected from a group of:
normal saline, which is 0.9% NaCl in water,
half saline, which is 0.45% NaCl in water,
D5W which is 5% dextrose in water, and
lactated ringer's solution (LR).

13. The method of claim 1, wherein the data input on the IV fluid infusion is a volume of the IV fluid infusion.

14. The method of claim 1, wherein the data input on the IV fluid infusion is a start time of the IV fluid infusion.

15. A blood contamination detection apparatus, comprising:
a computer-based contamination determining module configured to receive:
one or more baseline quantified test results on an uncontaminated blood sample obtained from a patient; and
one or more quantified test results for one or more analytes of a blood sample or a blood component; and
data input on a type of IV fluid infusion that the patient has received that is able to affect the one or more quantified test results, and
wherein the computer-based contamination determining module comprises a contamination determining routine executable on a computer and configured to compare differences between the one or more baseline quantified test results and the one or more quantified test results against preset threshold difference values and provide a contamination output.

16. The blood contamination detection apparatus of claim 15, wherein the preset threshold difference values are preset individually for each of the one or more analytes.

17. The blood contamination detection apparatus of claim 15, wherein the one or more analytes comprises glucose, sodium, chlorine, potassium, calcium, lactate, creatinine, and/or BUN.

18. The blood contamination detection apparatus of claim 15, wherein the contamination output provides a contamination probability.
